(19) United States
(12) Patent Application Publication
(10) Pub. No.: US 2025/0049702 A1
(43) Pub. Date: Feb. 13, 2025

(54) INTRANASAL DELIVERY OF CANNABIDIOL TO TREAT CENTRAL NERVOUS SYSTEM DISORDERS

(71) Applicant: Nose to Brain Therapeutics LLC, Beachwood, OH (US)

(72) Inventors: Thomas Irvin Bradshaw, Wynnewood, PA (US); Maria Jestl Bradshaw, Wynnewood, PA (US)

(21) Appl. No.: 18/928,283

(22) Filed: Oct. 28, 2024

Related U.S. Application Data
(63) Continuation of application No. 18/244,391, filed on Sep. 11, 2023, now abandoned.
Application No. 18/244,391 is a continuation of application No. 16/837,213, filed on Apr. 1, 2020, now abandoned.
(60) Provisional application No. 62/919,873, filed on Apr. 2, 2019.

(51) Int. Cl. A61K 9/00; A61K 9/14; A61K 31/05

(52) U.S. Cl. CPC A61K 9/0043; A61K 31/05; A61K 9/14

(57) ABSTRACT

Methods of treating central nervous system (CNS) conditions associated with oxidative stress are provided by delivering Cannabidiol (CBD) to the upper third of the nasal passage of a subject such that it transits the blood brain barrier to treat various brain conditions.

### CROSS-REFERENCE TO RELATED APPLICATIONS

This application is a continuation of U.S. patent application Ser. No. 18/244,391, filed Sep. 11, 2023, which is a continuation of U.S. patent application Ser. No. 16/837,213, filed Apr. 1, 2020, which claims priority to U.S. Provisional Patent Application No. 62/919,873, filed Apr. 2, 2019, which are each incorporated by reference in their entirety.

### TECHNICAL FIELD

The systems, methods, and compositions provided below generally relate to providing Cannabidiol (CBD) to the brain via delivery to the upper third of the nasal passage. More particularly, the systems, methods, and compositions provided below relate to treating various brain diseases through the delivery of CBD to the brain through the upper third of the nasal passage.

### BACKGROUND

CBD is known to have interesting pharmacologic properties. For example, CBD is the active ingredient in a recently registered drug, EPIDIOLEX, an oral solution of CBD for the treatment of seizures associated with two specific forms of epilepsy. The endogenous cannabinoid system (ECS) within the human body is responsible for neuromodulation, synaptic plasticity, and development of the central nervous system and peripheral nervous system. It consists of enzymes, cannabinoid receptors such and CB1 and CB2. CBD has minimal binding affinity to CB1 and CB2 but influences several endocannabinoid molecular signaling systems, their receptors and ion channels. CBD is metabolized in the human body by hydroxylation to form its acidic metabolites. This process is carried out with the help of hepatic P450 enzymes. FIG. 1 summarizes the functional properties exhibited by CBD at the cellular and molecular levels.

The oral bioavailability of CBD has been reported to be between 6-13%. Although it transits the blood brain barrier, because of its poor bioavailability, only a small percent orally dosed will be available to reach the brain. Furthermore, oral mucosal sprays have shown poor Cmax when compared to IV administration, as shown in FIG. 2.

### BRIEF DESCRIPTION OF THE DRAWINGS

The present disclosure will be more readily understood from a detailed description of some example embodiments taken in conjunction with the following figures:

FIG. 1 summarizes the functional properties exhibited by CBD at the cellular and molecular levels.

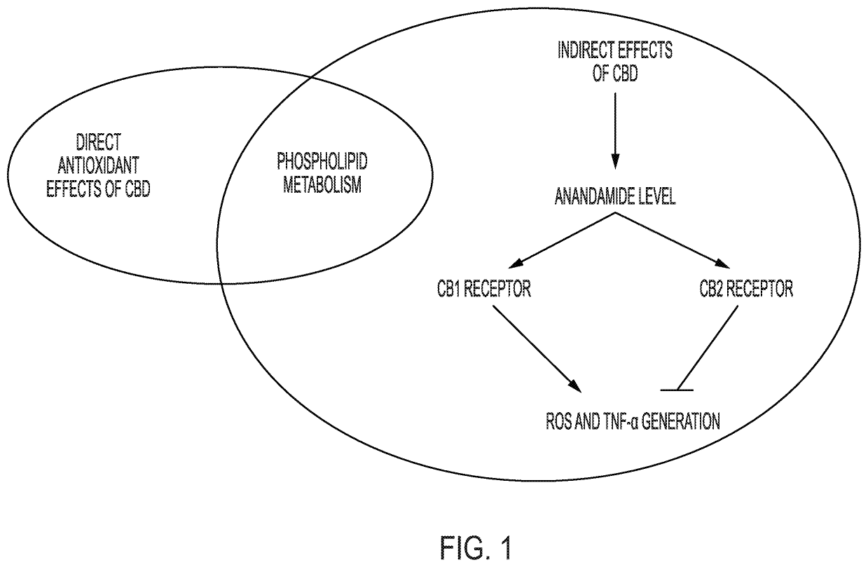

FIG. 2 depicts the dose versus C_{MAX} of CBD by various delivery routes.

FIG. 3 depicts the plasma concentration time profiles of CBD 200 μg/kg after IV (n=4) and IV (n=3) in rats.

### DETAILED DESCRIPTION

Various non-limiting embodiments of the present disclosure will now be described to provide an overall understanding of the principles of the structure, function, and use of the systems, methods, and compositions as disclosed herein. One or more examples of these non-limiting embodiments are illustrated in the accompanying drawings. Those of ordinary skill in the art will understand that systems, methods, and compositions specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the systems, methods, and compositions described herein. None of the features or components shown in the drawings or discussed below should be taken as mandatory for any specific implementation of any of these systems, methods, and compositions unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

The present disclosure generally improves the bioavailability of CBD administered to treat brain related indications such as Parkinson's disease, Attention Deficit Disorder (ADD), Attention Deficit Hyperactivity Disorder (ADHD), and other brain conditions, such as depression, autism disease, autism spectrum diseases and chronic fatigue syndrome for example. This improved brain bioavailability can be achieved by direct nose to brain delivery by means of delivery of the CBD to the upper third of the nasal passage. In so doing, the drug transits the blood brain barrier and travels directly to the brain. Conventional nasal sprays containing CBD do not deliver the CDB to the upper third of the nasal passage. Instead such conventional nasal sprays deliver the CBD to the blood stream via the nasal mucosa thereby resulting in poor bioavailability to the brain. Thus, the present disclosure provides improvements over conventional nasal sprays containing CBD.

Preclinical and clinical data has been accumulated to support the use of CBD in the central nervous system (CNS) for acute and chronic neurological conditions (e.g., Parkinson's disease, ADD, ADHD), as well as neuropsychiatric disorders (anxiety, depression, and schizophrenia). However, the concentrations required for CBD to mediate its salutary effects in the CNS in preclinical models require IV administration. Part of the challenge in moving CBD into human studies has been the lack of understanding of how to get sufficient bioavailability to achieve effectiveness, especially in the brain.

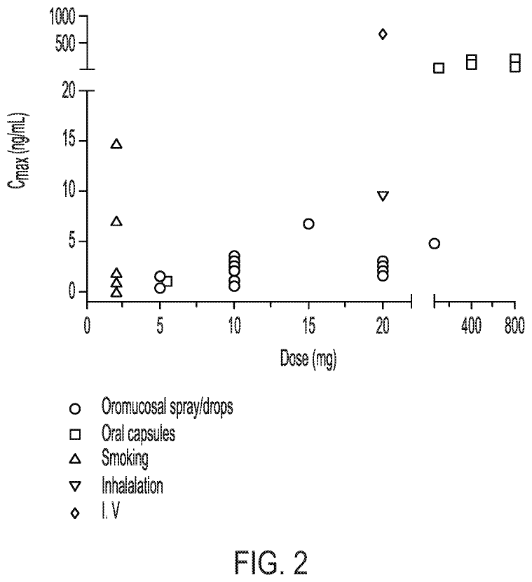

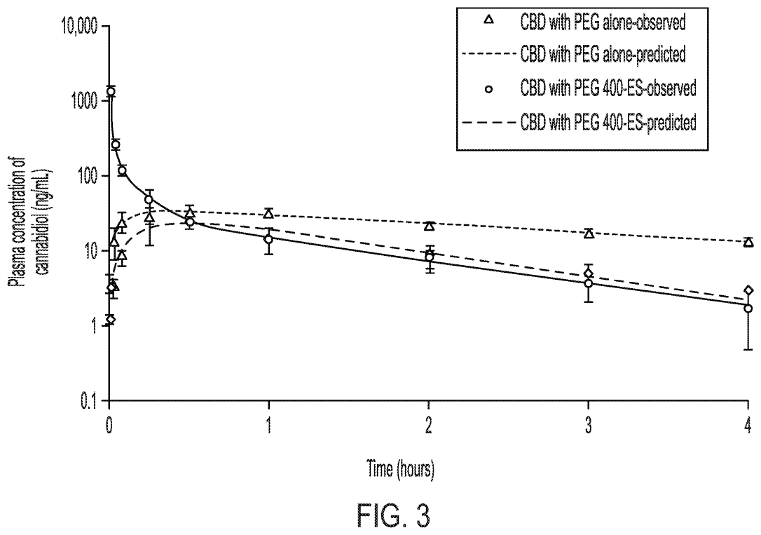

In accordance with the present disclosure, brain disorders can be targeted by delivering CBD intranasally to the upper third of the nasal passage and, thereby, achieving direct nose to brain delivery. Notably, neither oral administration nor nasal mucosal delivery achieve direct to brain delivery, but rather result in metabolism of CBD via the blood stream prior to delivery to the brain, as shown in FIG. 2. FIG. 3 depicts the plasma concentration time profiles of CBD 200 μg/kg after IV (n=4) and IN (n=3) in rats, as provided by Cannabidiol Bioavailability After Nasal And Transdermal Application: Effect of Permeation Enhancers Intranasal And Transdermal Delivery of Cannabidiol; Kalpana S. Paudel, Dana C. Hammell Remigius U. Agu1, Satyanarayana Valivetil, and Audra L. Stinchcomb, Drug Development and Industrial Pharmacy, 2010; 36(9): 1088-1097.

Thus, in accordance with the present disclosure, new routes of administration of CBD can be used, such as intranasal delivery, that can enhance the speed and quantities of CBD that reaches the brain to treat brain conditions, disease and interdict injury, yet with fewer potential side effects. In accordance with various embodiments, one or more of the administering steps in a treatment method comprise intranasal administration.

The systems, methods, and compositions described herein can be used to treat various CNS conditions, such as conditions associated with oxidative stress, endoplasmic reticulum stress, excitotoxic stress, among others. In some embodiments, the condition is a neurodegenerative condition, such as, for example, Alzheimer's disease, Parkinson's disease, Huntington's disease, multisystem atrophy, multiple sclerosis, amyolotrophic lateral sclerosis, cerebral ischemia, seizure disorders, schizophrenia, Friedreich's ataxia, progressive supranuclear palsy, prions, Down's syndrome, ataxia, tardive dyskinesia, or aging. In some embodiments, the condition is a neuropsychiatric disorder, such as, without limitation, schizophrenia, bipolar disorder, or depression. The present disclosure also provides CBD, as described herein, for use in treating CNS condition in accordance with any of the methods described herein.

CBD is being tested for many neuropsychiatric disorders. The present disclosure provides methods for treating a central nervous system (CNS) condition associated with excitotoxicity and/or oxidative stress in a subject in need thereof (e.g., a human subject). As used herein, “treat”, “treating” and/or “treatment” refers to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

As used herein, “therapeutically effective amount” refers to the amount of the therapeutic agent being administered that will relieve, to some extent, one or more of the symptoms of the disorder being treated. A therapeutically effective amount of an agent for halting or repairing neurodegeneration, for example, may result in reduced loss of neurons and/or their supporting cells (e.g., oligodendrocytes), enhancement of repair mechanisms, restored functionality, stimulated regeneration, glial reconstruction, or other clinical endpoints desired by healthcare professionals. Vision, motor, cognitive/mood, sensory, and pain impairments may be targets for treatment.

As is known in the art, the blood-brain barrier (BBB) is a persistent obstacle for the local delivery of therapeutic agents to the central nervous system (CNS). Thus, in accordance with various embodiments, advanced medical imaging techniques, such as MRS (Magnet Resonance Spectroscopy), can be utilized to monitor and determine the delivery of the CBD to the brain.

Various methods described herein can comprise administration of CBD alone or in combination with one or more other agents. In some embodiments, the CBD may be co-administered or formulated with another agent for the treatment of a CNS condition associated with oxidative, endoplasmic reticulum (ER), or excitotoxic stress. The other agent may be selected from, for example, glutathione or a prodrug thereof (e.g., glutathione ethyl ester), prostaglandin PGE2, an activator of nuclear factor (erythroid-derived 2)-like 2 (NFE2L2 or Nrf2), a lipid peroxidation inhibitor (e.g., vitamin E and its analogs or ferrostatin), an antioxidant (e.g., CoQ10), a transcription inhibitor, sodium selenite, a selenocysteine peptide, an iron chelator, an ERK1/2 inhibitor, a RIPK inhibitor, adaptaquin, or necrostatin-1. Combination with Schwann cell or other appropriate cell transplantation also is contemplated.

In some embodiments, the CNS condition associated with oxidative stress treated by the currently provided methods is a neurodegenerative condition (e.g., Alzheimer's disease, Parkinson's disease, Huntington's disease, multisystem atrophy, multiple sclerosis, amyolotrophic lateral sclerosis, cerebral ischemia, seizure disorders, schizophrenia, Friedreich's ataxia, progressive supranuclear palsy, prions, Down's syndrome, ataxia, tardive dyskinesia, or aging), or a neuropsychiatric condition (e.g., schizophrenia, bipolar disorder, or depression).

The present disclosure also provides a pharmaceutical composition comprising CBD as an active ingredient for treating a CNS condition associated with oxidative stress. The pharmaceutical composition may also comprise one or more pharmaceutically acceptable excipients. The term “excipient” is used herein to describe any ingredient other than the selenocysteine-comprising compound(s) described herein. The choice of excipient(s) will, to a large extent, depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as emulsifying agents, penetration enhances, preservatives, or buffers, which enhance the shelf life or effectiveness of the antibody.

Example pharmaceutical compositions and methods for their preparation can be found, for example, in Remington's Pharmaceutical Sciences, 19th Edition (Mack Publishing Company, 1995). Pharmaceutical compositions are preferably manufactured under GMP (good manufacturing practices) conditions.

Pharmaceutical compositions in accordance with the present disclosure can be prepared, packaged, or sold in bulk, as a single unit dose or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The CBD is to be administered intranasally to the upper third of the nasal passage, typically in the form of a dry powder. The dry power can be administered alone, as a mixture, or as a mixed component particle, for example, mixed with a suitable pharmaceutically acceptable excipient. In some embodiments, the CBD can be administered intranasally from a dry powder inhaler; as an aerosol spray from a pressurised container, pump, spray, atomiser (such as an atomizer using electrohydrodynamics to produce a fine mist), or nebulizer, with or without the use of a suitable propellant; or as nasal drops. The pressurized container, pump, spray, atomizer, or nebulizer can contain a solution or suspension of CBD that further comprises, for example, a suitable agent for dispersing, solubilizing, or extending release of the compound, and may comprise a propellant(s) as solvent.

Prior to use in a dry powder or suspension formulation, the compound can be micronized to a size suitable for delivery by inhalation (typically less than about 5 microns, for example). The compound can be micronized by any appropriate comminuting method, such as spiral jet milling, fluid bed jet milling, supercritical fluid processing to form nanoparticles, high pressure homogenization, spray drying, and so forth.

Moreover, capsules, blisters and cartridges for use in an inhaler or insufflator can be formulated to contain a powder mix of the compound, such as a suitable powder base and/or a performance modifier.

A suitable solution formulation for use in an atomizer using electrohydrodynamics to produce a fine mist may contain a suitable dose of the compound per actuation, and the actuation volume may, for example, vary from 1 μL to 100 μL.

Suitable flavours, such as menthol and levomenthol, or sweeteners, such as saccharin or saccharin sodium, can be added to those formulations intended for inhaled/intranasal administration. Moreover, formulations for intranasal administration may be formulated to be immediate and/or modified release. Modified release formulations include delayed-, sustained-, pulsed-, controlled-, targeted- and programmed-release.

In the case of dry powder inhalers and aerosols, the dosage unit may be determined by means of a valve which delivers a metered amount. Dosage units in accordance with the disclosure are typically arranged to administer a metered dose or “puff” of a compound. The overall daily dose can typically be administered in a single dose or, more usually, as divided doses throughout the day. In various embodiments, CBD is administered intranasally.

Further, it is be understood by one skilled in the art that CBD described herein may be used in a method of treatment as described herein, may be for use in a treatment as described herein, and/or may be for use in the manufacture of a medicament for a treatment as described herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to the particular uses contemplated. The scope is, of course, not limited to the examples set forth herein, but can be employed in any number of applications and equivalent devices by those of ordinary skill in the art. Rather, it is hereby intended that the scope of the invention is to be defined by the claims appended hereto.

We claim:

1. A method of treating a central nervous system (CNS) condition in a subject, comprising:
administering intranasally a composition directly to a brain of the subject through direct nose-to-brain delivery using a pressurized container, pump, spray, atomizer, or nebulizer, the composition comprising a therapeutically effective amount of Cannabidiol (CBD), wherein the composition is delivered to an upper third of a nasal passage and travels directly to the brain of the subject, thereby providing greater bioavailability of CBD to the brain compared to oral administration and reducing potential drug-drug interactions associated with systemic delivery methods.

2. The method of claim 1, wherein the composition is administered using an electrohydrodynamic atomizer that produces a fine mist.

3. The method of claim 2, wherein the electrohydrodynamic atomizer delivers an actuation volume between 1 μL to 100 μL.

4. The method of claim 1, wherein the bioavailability of CBD to the brain is greater than the 6-13% bioavailability achieved through oral administration.

5. The method of claim 1, wherein the CBD is provided in a dry powder.

6. The method of claim 1, wherein the CBD is provided in a solution or suspension.

7. The method of claim 6, wherein the composition further comprises any of a solvent, a propellant, a flavour, and a sweetener.

8. The method of claim 1, wherein the CNS condition is a brain injury.

9. The method of claim 1, wherein the CNS condition is a neurodegenerative condition.

10. The method of claim 9, wherein the neurodegenerative condition is Alzheimer's disease.

11. The method of claim 9, wherein the neurodegenerative condition is Parkinson's disease or a seizure disorder.

12. The method of claim 1, wherein the CNS condition is epilepsy.

13. The method of claim 1, wherein the CNS condition is a neuropsychiatric disorder.

14. The method of claim 13, wherein the neuropsychiatric disorder is any of schizophrenia, anxiety, bipolar disorder, attention deficit disorder (ADD), attention deficit hyperactivity disorder (ADHD), and depression.

15. The method of claim 1, wherein the CNS condition is associated with any of oxidative stress, endoplasmic reticulum stress, and excitotoxic stress.

16. A method of treating a central nervous system (CNS) condition in a subject, comprising:
administering intranasally a composition directly to a brain of the subject through direct nose-to-brain delivery, the composition comprising a therapeutically effective amount of Cannabidiol (CBD) in a solution or suspension form, delivered via an aerosol spray to an upper third of a nasal passage, wherein the composition travels directly to the brain of the subject, thereby providing greater bioavailability of CBD to the brain compared to oral administration and reducing potential drug-drug interactions associated with systemic delivery methods.

17. The method of claim 16, wherein the CNS condition is selected from the group consisting of a brain injury, a neurodegenerative condition, or epilepsy.

18. The method of claim 17, wherein the CNS condition is selected from the group consisting of a neuropsychiatric disorder selected from the group consisting of schizophrenia, anxiety, bipolar disorder, attention deficit disorder (ADD), attention deficit hyperactivity disorder (ADHD), and depression.

19. A method of treating a central nervous system (CNS) condition in a subject in need thereof, comprising of:
administering intranasally a composition directly to a brain of the subject through direct nose-to-brain delivery using a pressurized container, pump, spray, atomizer, or nebulizer, the composition comprising a therapeutically effective amount of Cannabidiol (CBD) and a pharmaceutically acceptable excipient, delivered to an upper third of a nasal passage, wherein the composition travels directly to the brain of the subject, thereby providing greater bioavailability of CBD to the brain compared to oral administration and reducing potential drug-drug interactions associated with systemic delivery methods, wherein the pharmaceutically acceptable excipient is selected from the group consisting of a solvent, a dispersion media, a coating, an antibacterial or antifungal agent, an isotonic agent, an absorption delaying agent, an emulsifying agent, a penetration enhancer, a preservative, a buffer, water, saline, phosphate buffered saline, glycerol, ethanol, a sugar, a polyalcohol, a salt, and a combination thereof.

20. The method of claim 19, wherein the bioavailability of CBD to the brain is greater than the 6-13% bioavailability achieved through oral administration.
